(19) United States
(12) Patent Application Publication
(10) Pub. No.: US 2005/0008691 A1
(43) Pub. Date: Jan. 13, 2005

(54) Bicalutamide compositions

(76) Inventors: Arturo Siles Ortega, Barcelona (ES); Joan Cucala Escoi, Barcelona (ES)

Correspondence Address: MARK R. BUSCHER, P.O. BOX 161, CATHARPIN, VA, 20143, US

(21) Appl. No.: 10/842,632

(22) Filed: May 11, 2004

Related U.S. Application Data
(60) Provisional application No. 60/470,224, filed on May 14, 2003.

(51) Int. Cl. A61K 9/48; A61K 9/20; A61K 9/14; A61K 31/277

(52) U.S. Cl. 424/451; 424/464; 514/522

(57) ABSTRACT

A bicalutamide pharmaceutical composition having a high content of bicalutamide is provided. The composition can be made from micronized bicalutamide in order to enhance the speed of dissolution and is preferably made from a granulate of bicalutamide that contains at least 50 (w/w)% of bicalutamide

This application claims the benefit of priority from under 35 U.S.C. 119(e) from U.S. Provisional Application Ser. No. 60/470,224, filed May 14, 2003, the entire contents of which are incorporated herein by reference.

### BACKGROUND OF THE INVENTION

The present invention relates to bicalutamide pharmaceutical compositions.

Bicalutamide is the common name for the compound 4′-cyano-3-((4-fluorophenyl)sulfonyl)-2-hydroxy-2-methyl-3′-(trifluoromethyl)propionanilide, and is represented by the formula (1): 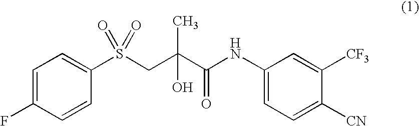
This compound can also be named N-(4-cyano-3-trifluoromethylphenyl)-3-(4-fluorophenylsulfonyl)-2-hydroxy-2-methyl-propionamide (see for instance TUCKER et al., J. Med. Chem., 31:954-959 (1988) for the former nomenclature and WO 01/00608 for the latter nomenclature).

Bicalutamide and related acylanilides have been disclosed in EP 100172 and corresponding U.S. Pat. No. 4,636,505 as pharmaceutically active compounds that possess antiandrogenic activity. Such compounds are useful, e.g., in treating prostate cancer. A bicalutamide pharmaceutical product is approved in many countries of the world under the brand name CASODEX (AstraZeneca). In marketed pharmaceutical compositions, bicalutamide is used as a racemate.

Generally, the marketed bicalutamide tablets comprise 50 or 150 mg of bicalutamide. Furthermore, inactive ingredients, such as lactose, polyvinylpyrrolidone, magnesium stearate, and carboxymethylstarch sodium, are used in the tablet core. The core is coated by a standard film coat comprising hypromelose, macrogol 300, and titanium dioxide. The tablet core is made by a wet granulation process, wherein industrial methylated spirit is used as a liquid vehicle for manufacturing the tablets. A tablet comprising 50 mg of bicalutamide has a total weight of about 128 mg, and the diameter of the tablet is about 6 mm. A tablet comprising 150 mg of bicalutamide has a total weight of about 384 mg, and the diameter of the tablet is about 9 mm.

WO 95/19770, which relates to the use of optically pure bicalutamide, describes further bicalutamide compositions. Example 1 of WO 95/19770 involves filling capsules with a blend of 10-50 mg of the R-enantiomer of bicalutamide, 35 mg of cornstarch, 1 mg of magnesium stearate, and a significant amount of lactose. Example 2 of WO 95/19770 involves making a tablet using water to make a granulate comprising bicalutamide.

WO 02/067893, WO 02/080902, and GB 2 372 444 disclose solid dispersions that include bicalutamide. Preparation of these solid dispersions generally includes evaporation of a solvent to leave a solid residue of the previously dissolved binder and bicalutamide. WO 02/067893 and WO 02/080902 also disclose that the solid dispersion may be used in forming capsules or tablets.

The relatively high amount of inactive ingredients, approximately 61% of the total tablet mass in the commercial product, leads to a need to produce tablets of a larger size. These large tablets may be difficult to swallow for some patients. It would be desirable to provide capsule or smaller tablet forms, which preferably had the same or similar release profile as the known commercial products.

### SUMMARY OF THE INVENTION

The present invention relates to the discovery that pharmaceutical compositions containing high amounts of bicalutamide can be formed that exhibit good drug release properties/profiles. Further, that micronized bicalutamide is advantageous for forming such compositions as well as high-load intermediate compositions especially granulates.

Thus, a first aspect of the present invention relates to a solid oral dosage form comprising at least 40% bicalutamide and at least one pharmaceutically acceptable excipient. Preferably the bicalutamide used to make such an oral dosage form is micronized bicalutamide.

A further aspect of the invention relates to a granulate, comprising at least 50% bicalutamide and at least one pharmaceutically acceptable excipient. The bicalutamide is preferably, though not necessarily, micronized bicalutamide. The granulate can be used to form a pharmaceutical composition such as a capsule or tablet. Typically the pharmaceutical composition comprises the granulate and an auxiliary excipient in an amount of up to 25% of the pharmaceutical composition.

Another aspect of the invention relates to the use of the bicalutamide compositions of the invention in treating an androgen disorder. For instance, a process of treating an androgen disorder, which comprises administering an effective amount of any of the above-mentioned bicalutamide-containing oral dosage forms or pharmaceutical compositions to a patient in need of such treatment.

A further aspect of the present invention relates to a process that comprises granulating a mixture comprising bicalutamide and at least one pharmaceutically acceptable excipient to form a granulate comprising at least 50 (w/w)% of bicalutamide. The granulating can be carried out by wet granulation, dry granulation or melt granulation. In a specific aspect, the granulation process is performed in the absence of an organic solvent. Preferably the bicalutamide used in forming the mixture is micronized bicalutamide.

### BRIEF DESCRIPTION OF THE DRAWINGS

FIG. 1 is a dissolution profile of bicalutamide tablets A, made from bicalutamide having a specific surface area of 4.6 m²/g, having a hardness of 28N.

FIG. 2 is a dissolution profile of bicalutamide tablets A, made from bicalutamide having a specific surface area of 4.6 m²/g, having a hardness of 77N.

FIG. 3 is a dissolution profile of bicalutamide tablets B, made from bicalutamide having a specific surface area of 0.5 m 2/g, having a hardness of 54N.

FIG. 4 is a dissolution profile of bicalutamide tablets C, made from bicalutamide having a specific surface area of 3.0 m²/g, having a hardness of 28N.

FIG. 5 is a dissolution profile of bicalutamide tablets D, made from bicalutamide having a specific surface area of 1.6 m²/g and SDS inside the granulate, and having a hardness of 34N.

FIG. 6 is a dissolution profile of bicalutamide tablets E, made from bicalutamide form II, having a hardness of 34N.

### DESCRIPTION OF THE INVENTION

The present invention relates to pharmaceutical compositions having at least 40% bicalutamide and at least one pharmaceutically acceptable excipient as well as to ingredients and intermediate compositions thereof. By providing high loading of the bicalutamide, i.e. amounts of at least 40%, the overall size of the finished dosage form can be reduced. Preferably the amount of bicalutamide is within the range of 40% to 90%, more preferably 50% to 80%.

The bicalutamide used in the present invention can be any form of bicalutamide, including racemic bicalutamide, single enantiomers of bicalutamide, mixtures thereof as well as crystalline or amorphous forms. Normally crystalline forms are preferred. In this regard, crystalline racemic bicalutamide is generally preferred, such as Form I and/or Form II crystalline bicalutamide as discussed in U.S. provisional patent application No. 60/413,765, filed Sep. 27, 2002, which is incorporated in its entirety herein by reference. In general, Form II is obtained by precipitating at higher temperatures, such as 30° C. or higher and optionally in the presence of a Form II seed crystal.

The bicalutamide is normally formulated into a pharmaceutical composition as solid particles, typically having an average particle size of 0.1 to 100 microns, more typically 1 to 50 microns. Preferably, however, the bicalutamide is employed in a micronized state; i.e., as fine particles, in forming the pharmaceutical composition. As used herein, “micronized” means that the bicalutamide particles satisfy at least one of the following parameters: (i) an average particle size of 0.1 to 20 microns, preferably 1 to 10 microns, more preferably 2 to 8 microns; (ii) a density of 1.3 to 1.6 mg/ml; or (iii) a specific surface area of at least 0.6 m²/g, preferably at least 1.2 m²/g, more preferably at least 3 m²/g. In theory, each of these properties is reflective of the same fact, namely that the bicalutamide particles are of a fine size. While particle size is seemingly the most direct measurement, odd shaped particles, such as rods, and/or measuring techniques can sometimes distort the result. Density and surface area are generally easier to measure and generally vary in proportion to particle size. Accordingly, as long as at least one of the three parameters is satisfied by a sample of bicalutamide particles, then the bicalutamide is micronized. For clarity, density refers to true density and is normally measured by a pycnometer, such as a helium pycnometer. Preferably the micronized bicalutamide satisfy at least two of the parameters, more preferably it satisfies all three parameters.

In some embodiments it is desirable to have a relatively uniform particle size. A uniform particle size means that at least 67% of the population, more preferably at least 90% of the population, falls within +/−10 microns of the average particle size.

The particulate bicalutamide is generally of high purity given its pharmaceutical utility and is typically at least 99% pure. The bicalutamide molecule can be made by synthetic routes known in the art. To obtain micronized bicalutamide, any technique that produces the desired fine particle size can be used. For example, a milling/micronizing process using, e.g., a Jet-Mill JP mill, can be used to convert bulk bicalutamide into micronized bicalutamide. Alternatively, bicalutamide of a desired particle size may be obtained by controlling the conditions during precipitation from a solution and/or by spray drying or crystallization in an ultrasonic bath.

While the bicalutamide, especially micronized bicalutamide, can be directly combined with other pharmaceutically acceptable excipients to form a pharmaceutical composition such as a tablet or capsule, etc., it is generally preferred to first form a granulate containing the bicalutamide. The granulate, which is generally free flowing, includes bicalutamide in amounts of at least 40%, preferably at least 50%, more preferably at least 60%, and in some embodiments at least 80%. A preferred range is 60-90% of the total mass of the granulate is bicalutamide. The granulate contains at least one pharmaceutically acceptable excipient, especially a binder, a disintegrant, a wetting surface-active agent, and/or a melt granulation excipient, but is not limited thereto. The binder, such as polyvinylpyrrolidone, may be present in an amount of 1-35% of the total mass of the granulate. The disintegrant, such as sodium starch glycolate or crosspovidone, may be present in an amount of 1-25% of the total mass of the granulate. The wetting surface-active agent, such as sodium dodecyl sulfate (SDS) or d-α-tocopheryl polyethylene glycol 1000 succinate (vitamin E TPGS), may be present in an amount up to 2%, such as from critical micellar concentration (CMC) to 2% of the total mass of the granulate. The melt granulation excipient may be chosen from glyceryl esters of fatty acids (Precirol, Compritol), polyethyleneglycols (Macrogols) or their glyceryl-derivatives (Gelucires). Additionally, the granulate may contain other suitable auxiliary excipients and traces of water.

The granulate is generally comprised of granules having an average particle size of 0.01 mm to 1.5 mm, more typically 0.1 mm to 0.5 mm. The granulate may be produced by applying essentially any known granulation technique to a mixture comprising bicalutamide and one or more pharmaceutically acceptable excipients to thereby form a granulate. Examples of suitable granulation techniques include wet granulation, dry granulation, and melt granulation.

Wet granulation, which is generally carried out with water, can be uneconomical or impractical when applied to a highly hydrophobic drug, like bicalutamide, in a high loading concentration. This problem may be solved by adding an organic solvent which serves to decrease the dielectric constant of water, improve the wettability, and increase the solubility of the product. For example, an aqueous alcohol of concentration of 60 (v/v)% can be used in making a granulate comprising up to 90 (w/w)% of bicalutamide. The use of organic solvent, however, causes environmental and safety concerns and a wet granulation technique that does not use any organic solvent is preferable.

Surprisingly, it has been discovered that a granulate comprising at least 60% of bicalutamide can be made by performing granulation in an absence of an organic solvent. In a typical embodiment, bicalutamide is granulated with a binder, e.g., polyvinylpyrrolidone or fatty acid wax, and/or a wetting agent, e.g., sodium lauryl sulfate, and/or a disintegrant, in presence of water (e.g. wet granulation) or in total absence of solvents (e.g. dry granulation). Thus, granulates comprising up to 90 (w/w)% of bicalutamide can be produced without using organic solvents in the granulation process via wet granulation, dry granulation or melt granulation.

The wet-granulation process can comprise adding bicalutamide to a single-pot or similar equipment and mixing therewith a binder (e.g., polyvinylpyrrolidone) and/or wetting agent to form a mixture. Optionally, a filler and/or disintegrant can also be added and homogenized. The mixture is then granulated with sufficient water. Alternatively, the binder and wetting agent can be added as an aqueous granulating solution to the dry mixture of bicalutamide and other components.

After granules are formed by wet granulation, a drying step is generally performed. The drying step may include using a vacuum, microwave radiation, heating air, heating double-jacket, and/or gas flow (N₂ or air). The resulting granulate may be gently sieved to obtain a free flowing granulate.

Alternatively, the granulates may be formed by dry granulation, also known as compaction. The method can include forming a dry homogeneous mixture of bicalutamide with one or more excipients and passing the mixture through a roll-compactor to obtain ribbons. Suitable inert excipients useful in this process include binder, disintegrant, filler, and lubricant. The roll-compacted ribbons may then be milled and sized to a free-flowing granulated powder.

As an alternative to wet or dry granulation, melt granulation may be used. Melt granulation generally comprises mixing the bicalutamide with a melt granulation excipient and optionally additional excipients; melting the mixture up to melting temperature (e.g., generally below 75° C.), by means of microwaves, hot air, and/or a water-jacketed vessel, while stirring continuously; and cooling the product to a processing temperature suitable for extruding, milling, and/or sieving in order to form a granulate. A melt granulation excipient is a lipophilic matrix forming material that has a melting or softening point at 80° C. or less. Preferred melt granulation excipients are waxes and esters of fatty acids. Because of the relatively high melting point of bicalutamide, the bicalutamide does not normally melt during the melt granulation. Thus, like in wet and dry granulation, the solid state form, i.e. crystalline form and particle size, is generally preserved during the hot melt granulation as well. Indeed, it is preferred that a true dispersion (e.g. molecular dispersion) of bicalutamide in the melt granulation excipient is not formed.

Regardless of the granulation technique, the resulting high load bicalutamide granulate can be used to form a finished dosage form, especially a solid oral dosage form. Accordingly, the bicalutamide granulate of the present invention may be mixed in a suitable mixer, e.g., a free fall mixer, with auxiliary excipients, such as filler(s), disintegrant(s), lubricant(s), glidant(s), to provide an homogeneous mixture of desired properties and concentration of the active substance. Examples of the filler include lactose monohydrate and pregelatinized starch. An example of the disintegrant is sodium starch glycolate. An example of the lubricant is magnesium stearate. An example of the glidant is silicon dioxide. The amounts and type of the auxiliary excipients depend on the desired physical properties of the final composition and desired concentration of bicalutamide. Alternatively, the granulate may be suitable for direct filling into capsules or for making a sachet without adding any auxiliary excipient.

Preferably the amount of bicalutamide in the pharmaceutical composition, whether incorporated by a granulate or not, is at least 40%, more preferably 50% to 80%. In unit doses such as a single tablet or capsule the absolute amount of bicalutamide is preferably within the range of 20 mg to 200 mg, especially 50 mg, 75 mg, 100 mg, and 150 mg.

The pharmaceutical compositions of the present invention may or may not comprise lactose. Lactose, though being a common excipient in pharmaceutical compositions, may cause irritation in the stomach at sensitive patients. The compositions and processes of the invention allow lactose to be excluded from the bicalutamide dosage forms.

The granulate or mixture of granulate and auxiliary excipients can be directly encapsulated into capsules, such as hard gelatin-capsules, in a suitable capsule machine. The amount of bicalutamide, as a concentration, in the final composition for filling into capsules can be about 40-80 (w/w)%, such as 40-70 (w/w)%, including 45-50 (w/w)%, of the total capsule weight. The amount may be adjusted by selecting the relative amounts of the granulate and other inactive ingredients. Thus, the granulate may represent a concentrate of bicalutamide. Dilution may be made by means of, e.g., a filler, the amount of which can be selected so that the whole space of the capsule of a selected size is essentially filled by the final composition. For instance, a composition having a total mass of 126 mg, comprising 50 mg of bicalutamide, is appropriate for filling a capsule of size 4. Alternatively, a composition having a total mass of 300 mg, comprising 150 mg of bicalutamide, is appropriate for filling a capsule of size 1. Examples of the size of the capsule include 1, 2, 3, and 4. The capsules may be made from gelatin or HPMC (hydroxy propyl methyl cellulose).

In addition to capsules, the bicalutamide of the present invention, eitherper se as in direct compression or in granulate form as in wet or dry granulation, etc., may be used for making tablets. The tablets can include 60-90 (w/w)% of bicalutamide.

Additionally, the granulate may be filled into sachets. The sachets may be made and filled by essentially any sachet making and filling processes.

The pharmaceutical compositions of the present invention, especially the solid oral dosage forms, not only have a high load of bicalutamide, but preferably have a dissolution profile in vitro that includes at least 75% bicalutamide released at thirty minutes. For purposes of the present invention, an in vitro dissolution profile refers to the dissolution of bicalutamide when the composition is subjected to a dissolution study in 900 ml aqueous buffered system (pH 7) with 0.75 (w/v)% SDS using a USP apparatus 2 (paddles) at 50 RPM at 37° C. Preferably at least 80%, more preferably at least 90% of the bicalutamide is released from the pharmaceutical composition after 30 minutes have elapsed. More preferably the composition is bioequivalent in vivo to the commercially available bicalutamide tablet. In particular, by the present invention a bioequivalent capsule to the commercial tablet can be formed.

Any of the above described pharmaceutical compositions can be used to treat an androgen disorder, especially prostate cancer, by administering an effective amount thereof to a patient in need thereof.

Each of the patents and articles mentioned above are incorporated herein by reference in their entirety. The present invention is further described by the following non-limiting examples.

### EXAMPLES

### Example 1

### Bicalutamide Tablets

The composition of the tablets is shown in the following Table 1.

TABLE 1

| Ingredients | Tablets A | Tablets B-C | Tablets D |
|---|---|---|---|
| Bicalutamide | 50.0 mg | 50.0 mg | 50.0 mg |
| Lactose monohydrate | 61.0 mg | 61.0 mg | 59.0 mg |
| Povidone | 5.0 mg | 5.0 mg | 5.0 mg |
| Crosspovidone | 7.5 mg | | |
| Sodium starch glycolate | | 7.5 mg | |
| Sodium dodecyl sulphate | | | 2.5 mg |
| Magnesium stearate | 1.5 mg | 1.5 mg | 1.0 mg |
| Total | 125 mg | 125 mg | 125 mg |

Particle characteristics of the bicalutamide:
Tablets A: particle size 6.1 μm; SSA 4.6 m²/g; density 1.52 g/ml
Tablets B: particle size 106.5 μm; SSA 0.5 m²/g; density 1.62 g/ml
Tablets C: particle size 5.9 μm; SSA 3.0 m²/g; density 1.54 g/ml
Tablets D: particle size 3.9 μm; SSA 1.6 m²/g; density 1.52 g/ml

Tablets A)

Bicalutamide was mixed with the lactose monohydrate, povidone, and half of the disintegrant (crosspovidone) in an instrumented single-pot granulator (Mi-Mi-Pro, Pro-C-epT). Purified water was added (0.27 ml/g active substance), to obtain a wet granulated mass. The resulting mass was dried using a combination of microwave irradiation (50-200 W), vacuum (below 100 mb) and hot air (50-60° C.).

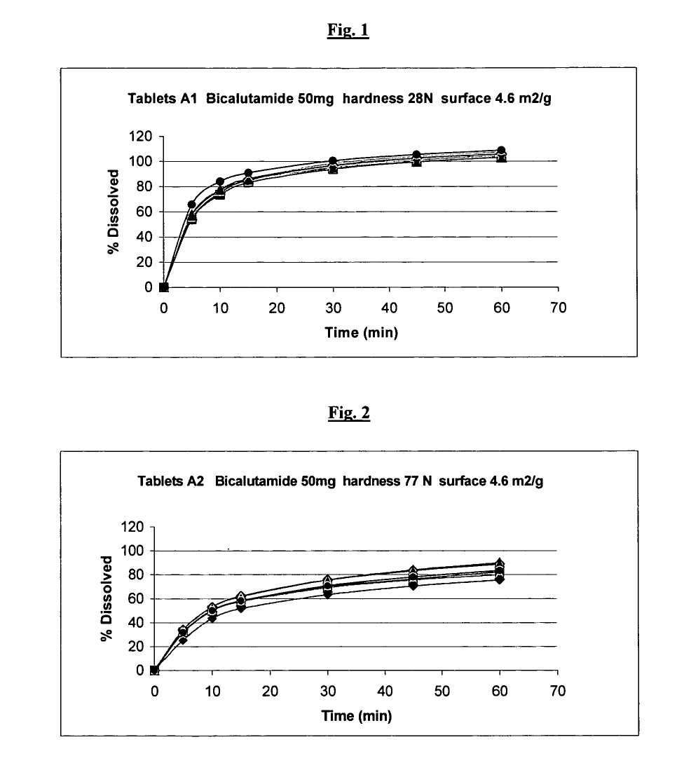

The dried product was then milled and sieved (through 500 μm mesh) until granules of the required size (below 500 μm) were obtained. The granulate was then mixed with the rest of the disintegrant and lubricant just before compression was performed in an eccentric instrumented Korsch EKO press machine at variable pressures (range 2.4-6.7 KN) with round punches of 6 mm diameter. The dissolution profile of the resulting tablets was tested multiple times by using the paddle method under the following conditions: 900 ml aqueous buffered system (pH 7) with 0.75 (w/v)% SDS using a USP apparatus 2 (paddles) at 50 RPM at 37° C. The results for 2.4 KN compressed tablets (A1) and 6.7 KN compressed tablets (A2) are shown in FIGS. 1 and 2.

Tablets A1 made under low pressure dissolved faster than Tablets A2 made under high pressure. In particular, FIG. 1 shows that tablets (Tablets A1) made under a tabletting force of 2.4 KN, providing tablets having a hardness of 28 N, exhibited 100% release in 30 minutes. FIG. 2 shows that the same composition compressed under a tabletting force of 6.7 KN, providing tablets of 77 N hardness, exhibited approximately 70% release in 30 minutes.

Tablets B)

Bicalutamide was mixed with the lactose monohydrate, povidone, and half of the disintegrant (sodium starch glycolate) in an instrumented single-pot granulator (Mi-Mi-Pro, Pro-C-epT). Purified water was added (0.25 ml/g active substance), to obtain a wet granulated mass. The resulting mass was dried using a combination of microwave irradiation (50-200 W), vacuum (below 100 mb) and hot air (50-60° C.).

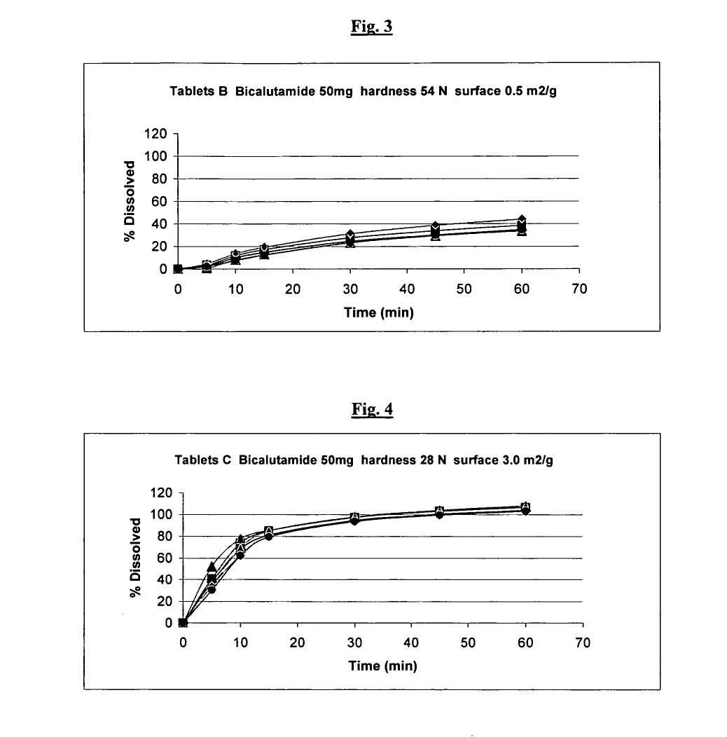

The dried product was then milled and sieved (through 500 μm mesh) until granules of the required size (below 500 μm) were obtained. The granulate was then mixed with the rest of the disintegrant and lubricant just before compression was performed in an eccentric instrumented Korsch EK0 press machine, at 8.0 KN pressure, with round punches of 6 mm diameter. The dissolution profile of the resulting tablets was tested multiple times by using the paddle method under the following conditions: 900 ml aqueous buffered system (pH 7) with 0.75 (w/v)% SDS using a USP apparatus 2 (paddles) at 50 RPM at 37° C. FIG. 3 represents the results of dissolution of B tablets having a hardness of 54N.

Tablets C)

Bicalutamide was mixed with the lactose monohydrate, povidone, and half of the disintegrant (sodium starch glycolate) in an instrumented single-pot granulator (Mi-Mi-Pro, Pro-C-epT). Purified water was added (0.25 ml/g active substance), to obtain a wet granulated mass. The resulting mass was dried using a combination of microwave irradiation (50-200 W), vacuum (below 100 mb) and hot air (40-50° C.).

The dried product was then milled (Böhle BTS turbosieve, equipped with 1.1 mm mesh) until granules of the required size (average below 500 μm) were obtained. The granulate was then mixed with the rest of the disintegrant and lubricant just before compression was performed in an eccentric instrumented Korsch EKO press machine at variable pressures (range 2.8-18.5 KN) with round punches of 6 mm diameter. The dissolution profile of the resulting tablets was tested multiple times by using the paddle method under the following conditions: 900 ml aqueous buffered system (pH 7) with 0.75 (w/v)% SDS using a USP apparatus 2 (paddles) at 50 RPM at 37° C. FIG. 4 represents the results of dissolution of C tablets having a hardness of 28N.

Tablets made from higher specific surface area bicalutamide dissolved faster than tablets made from lower specific surface area bicalutamide. Specifically, FIG. 3 shows that tablets (Tablets B) made from bicalutamide having a surface area of 0.5 m²/g exhibited approximately 25% release in 30 minutes. FIG. 4 shows that tablets (Tablets C) made from bicalutamide of a specific surface of 3.0 m²/g exhibited 100% release in 30 minutes.

Tablets D)

Bicalutamide was mixed with the lactose monohydrate, povidone, sodium dodecyl sulfate (SDS) and half of the disintegrant (crosspovidone) in an instrumented single-pot granulator (Mi-Mi-Pro, Pro-C-epT). Purified water was added (0.25 ml/g active substance), to obtain a wet granulated mass. The resulting mass was dried using a combination of microwave irradiation (50-200 W), vacuum (below 100 mb) and hot air (40-50° C.).

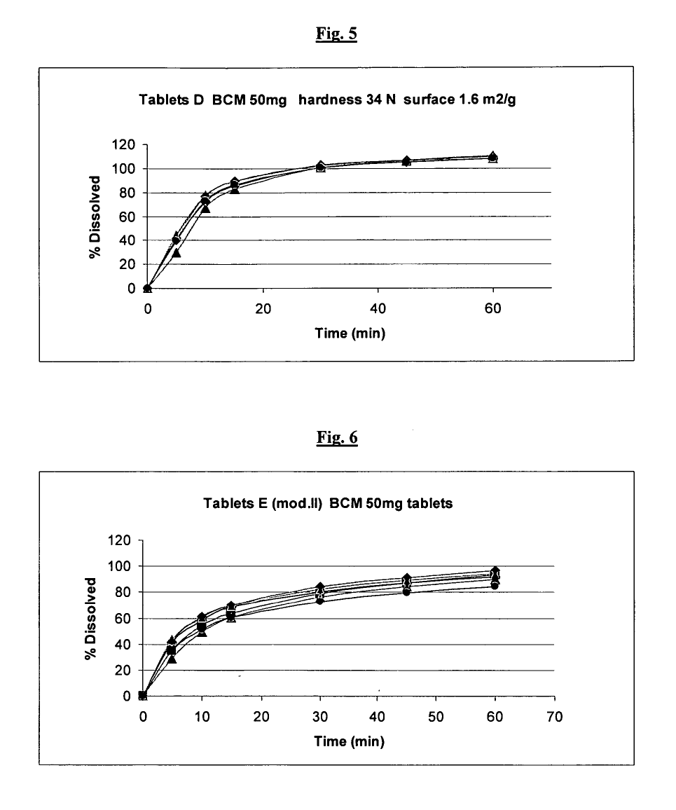

The dried product was then milled (Böhle BTS turbosieve, equipped with 1.1 mm mesh) until granules of the required size (average below 500 μm) were obtained. The granulate was then mixed with the rest of the disintegrant and lubricant just before compression was performed in an eccentric instrumented Korsch EK0 press machine at variable pressures (range 4.7-16.4 KN) with round punches of 6 mm diameter. The dissolution profile of the resulting tablets was tested multiple times by using the paddle method under the following conditions: 900 ml aqueous buffered system (pH 7) with 0.75 (w/v)% SDS using a USP apparatus 2 (paddles) at 50 RPM at 37° C. FIG. 5 represents the results of dissolution of D tablets having a hardness of 34N.

In view of the above, the dissolution profile of bicalutamide tablets is affected by particle size and/or surface area of the active substance and by the tabletting force of the tablet press.

### Examples 2-4

Particle Characteristics of the Bicalutamide:

Granulate A: particle size 6.7 μm; SSA 3.5 m²/g; density 1.47 g/ml
Granulate B: particle size 3.9 μm; SSA 1.6 m²/g; density 1.52 g/ml
Granulate C: particle size 3.9 μm; SSA 1.6 m²/g; density 1.52 g/ml

### Example 2

### Bicalutamide Granulate A (Made by Dry Granulation)

The composition of granulate (A) is shown in Table 2.

TABLE 2

| Ingredients | Granulate A | % |
|---|---|---|
| Bicalutamide | 120 g | 40.0 |
| Lactose monohydrate | 108 g | 36.0 |
| Silicified microcrystalline cellulose | 60 g | 20.0 |
| Sodium starch glycolate | 9.6 g | 3.2 |
| Magnesium stearate | 2.4 g | 0.8 |
| Total | 300 g | 100.0 |

The above materials were sieved, mixed for 30 minutes with a Turbula mixer, granulated through a roll-compactor (Chilsonator IR220, from Fitz-Patrick) at 11.9 KN/cm, and milled through a 0.5 mm mesh (Fitz-Mill, from Fitz-Patrick).

### Example 3

### Granulate Composition B (Made by Wet-Granulation)

The composition of granulate (B) is shown in Table 3.

TABLE 3

| Ingredients | Granulate B | % |
|---|---|---|
| Bicalutamide | 250 g | 83.33 |
| Povidone | 25 g | 8.33 |
| Sodium starch glycolate | 25 g | 8.33 |
| Total | 300 g | 100.0 |

The above materials were mixed and granulated with purified water (0.3 ml/g active substance) in a single-pot granulator MiMiPro (available from Pro-C-epT), The resulting mass was dried using a combination of microwave irradiation. (50-250 W), vacuum (below 100 mb) and hot air (50° C.) until the water activity was below 0.5, and sieved through a 0.25 mm mesh.

### Example 4

### Granulate Composition C (Made by Wet Granulation)

The composition of granulate (C) is shown in Table 4.

TABLE 4

| Ingredients | Granulate C | % |
|---|---|---|
| Bicalutamide | 225.00 g | 80.0 |
| Povidone | 22.50 g | 8.0 |
| Crosspovidone | 22.50 g | 8.0 |
| Sodium dodecyl sulfate | 11.25 g | 4.0 |
| Total | 281.25 g | 100.0 |

The above materials were mixed and granulated with purified water (0.25 ml/g active substance) in a single-pot granulator MiMiPro (available from Pro-C-epT), The resulting mass was dried using a combination of microwave irradiation (50-400 W), vacuum (below 100 mb) and hot air (40-50° C.) until the water activity was below 0.5, and sieved through a 0.25 mm mesh.

### Example 5

### Capsule Composition (of the Same Content as in Tablets B and C)

The composition of capsules A is shown in Table 5.

TABLE 5

| Ingredients | Capsules A | |
|---|---|---|
| Bicalutamide* | 50.0 | mg |
| Lactose monohydrate | 61.0 | mg |
| Sodium starch glycolate | 5.0* + 2.5 | mg |
| Povidone* | 5.0 | mg |
| Magnesium stearate | 1.5 | mg |
| Hard gelatin capsule No. | 4 | |
| Total | 125.0 | mg |

*= present in the granulate

Granulate (B) was mixed with the excipients not present in the granulate for 15 minutes by using a Turbula mixer. This blend was then filled into hard gelatin capsules.

The dissolution profile of the resulting capsules was tested by using the paddle method under the following conditions: 900 ml aqueous buffered system (pH 7) with 0.75 (w/v)% SDS using a USP apparatus 2 (paddles) at 50 RPM at 37° C.

More than 75% of the bicalutamide was dissolved within 30 minutes.

### Example 6

### Capsules Composition with SDS

The composition of Capsules B is shown in Table 6.

TABLE 6

| Ingredients | Capsules B | |
|---|---|---|
| Bicalutamide* | 50.0 | mg |
| Lactose monohydrate | 61.0 | mg |
| Povidone* | 5.0 | mg |
| Crosspovidone* | 5.0 | mg |
| Sodium dodecyl sulfate (SDS)* | 2.5 | mg |
| Magnesium stearate | 1.25 | mg |
| Silicon dioxide | 1.25 | mg |
| Hard gelatin capsule No. | 4 | |
| Total | 126.0 | mg |

*= present in the granulate

Granulate (C) was mixed with the excipients not present in the granulate for 10 minutes by using a Turbula mixer. This blend was then filled into hard gelatin capsules.

The dissolution profile of the resulting capsules was tested by using the basket method under the following conditions: 900 ml aqueous buffered system (pH 7) with 0.75 (w/v)% SDS using a USP apparatus 1 (baskets) at 100 RPM at 37° C.

More than 75% of the bicalutamide was dissolved within 30 minutes.

### Example 7

### Lactose-Free Capsules

The composition of capsules C is shown in Table 7.

TABLE 7

| Ingredients | Capsules C | |
|---|---|---|
| Bicalutamide* | 50.0 | mg |
| Pregelatinized starch | 61.0 | mg |
| Povidone* | 5.0 | mg |
| Crosspovidone* | 5.0 | mg |
| Sodium dodecyl sulfate (SDS)* | 2.5 | mg |
| Magnesium stearate | 1.25 | mg |
| Silicon dioxide | 1.25 | mg |
| Hard gelatin capsule No. | 4 | |
| Total | 126.0 | mg |

*= present in the granulate

Granulate (C) was mixed with the excipients not present in the granulate for 15 minutes by using a Turbula mixer. This blend was then filled into hard gelatin capsules.

The dissolution profile of the resulting capsules was tested by using the basket method under the following conditions: 900 ml aqueous buffered system (pH 7) with 0.75 (w/v)% SDS using a USP apparatus 1 (baskets) at 100 RPM at 37° C.

More than 75% of the bicalutamide was dissolved within 30 minutes.

### Example 8

### Capsule Composition (of the Same Content as in Tablets D)

The composition of capsules D is shown in Table 8.

TABLE 8

| Ingredients | Capsules D | |
|---|---|---|
| Bicalutamide* | 50.0 | mg |
| Lactose anhydrous | 59.0 | mg |
| Povidone* | 5.0 | mg |
| Crosspovidone* | 7.5 | mg |
| Sodium dodecyl sulfate (SDS)* | 2.5 | mg |
| Magnesium stearate | 1.0 | mg |
| Hard gelatin capsule No. | 4 | |
| Total | 125.0 | mg |

Same blend used for tablets D was then filled into hard gelatin capsules.

The dissolution profile of the resulting capsules was tested by using the paddle method under the following conditions: 900 ml aqueous buffered system (pH 7) with 0.75 (w/v)% SDS using a USP apparatus 2 (paddles) at 50 RPM at 37° C.

More than 75% of the bicalutamide was dissolved within 30 minutes.

### Examples 9-10

### Capsules of 150 mg Bicalutamide

The compositions of Examples 9 (Capsules E) and 10 (Capsules F) are shown in Table 9.

TABLE 9

| Ingredients | Capsules E | | Capsules F | |
|---|---|---|---|---|
| Bicalutamide* | 150.0 | mg | 150.0 | mg |
| Lactose monohydrate | 106.5 | mg | — | |
| Povidone* | 15.0 | mg | 15.0 | mg |
| Crosspovidone* | 15.0 | mg | 15.0 | mg |
| Sodium dodecyl sulfate (SDS)* | 7.5 | mg | 7.5 | mg |
| Magnesium stearate | 3.0 | mg | 1.5 | mg |
| Silicon dioxide | 3.0 | mg | — | |
| Hard gelatin capsule No. | 1 | | 3 | |
| Total | 300.0 | mg | 189.0 | mg |

*= present in the granulate

Granulate (C) was mixed with the rest of excipients for 15 minutes by using a Turbula mixer. This blend was then filled into hard gelatin capsules.

The dissolution profile of the resulting capsules was tested by using the basket method under the following conditions: 900 ml aqueous buffered system (pH 7) with 2 (w/v)% SDS using a USP apparatus 1 (baskets) at 100 RPM at 37° C.

More than 75% of the bicalutamide was dissolved within 30 minutes.

### Example 11 (Reference)

### Bicalutamide Forms

A) Form I

2.15 g of bicalutamide and 19.5 ml of ethyl acetate were transferred into a round bottomed 3 neck flask of 250 ml. The suspension was heated to reflux in an oil bath and stirred with magnetic stirrer and stirrer device. Reflux was maintained until a clear solution was obtained. The solution was cooled to 20° C. in a water bath while kept stirring. During cooling the bicalutamide crystallized. The suspension was then cooled to 5° C. in an ice bath. To the suspension 77 ml of petroleum ether (boiling range 40-70° C.) was added slowly. After addition, the suspension was stirred for 5 more minutes. The suspension was filtered over a p3-glass filter using reduced pressure. The solid material was washed with cold petroleum ether (boiling range 40-70° C.). The solid material was then dried at 60° C. and under vacuum overnight. According to DSC, IR and microscopy the obtained bicalutamide is crystalline form I.

B) Form II

1.0 g of bicalutamide Form I was transferred into a glass round bottomed flask of 100 ml. The flask was closed with a stopper and placed in an oil bath at 210° C. Within 5 minutes all active substance was molten (light yellow melt). Subsequently the flask was removed from the oil bath and the melt was allowed to cool to ambient temperature. The melt solidified to a glass. The flask was placed in an oil bath at 160° C. Within a few minutes the glass became liquid and crystals of bicalutamide form II were formed. The flask was removed from the oil bath after about 10 minutes and allowed to cool to ambient temperature. The solid mass was isolated and gently grinded to obtain particles, small enough for analysis.

C) Form II

In a 101 round-bottomed flask equipped with a mechanical stirrer, nitrogen inlet and an ice-acetone cooling bath, 31 of n-heptane was cooled to −5 to −10° C. 190 g of bicalutamide was dissolved in 2.521 of ethyl acetate at reflux. The cold, stirred n-heptane was seeded with 200 mg of bicalutamide form II. The hot bicalutamide solution in ethyl acetate was added slowly in 30 minutes to the cold stirred and seeded n-heptane. A white suspension was formed. The white suspension was stirred for 5 minutes and filtered over a glass-filter. Filtration took about 40 minutes. The white solid was washed with 2×200 ml cold n-heptane (0-4° C.). The solid was dried at air for 3 hours and was dried under vacuum at room temperature for 16 hours. Yield: 160 g of bicalutamide Form II; m.p. 189.7-191.6° C.; LOD: 0.1%; Purity: 99.78% (HPLC). The XRPD included the following peaks:

2θ (degrees)

11.555
13.015
16.150
18.110
24.300
25.195
25.570
25.800
26.685
29.870
33.610

### Examples 12-13

### Bicalutamide Form II Dosage Forms

The bicalutamide Form II produced according to the process of Example 11 was formulated into tablets and capsules.

### Example 12

### Form II Tablet Composition

The composition of Tablets E is shown in the following Table 10:

TABLE 10

| Ingredients | Tablets E |
|---|---|
| Bicalutamide | 50.0 mg |
| Lactose monohydrate | 61.0 mg |
| Povidone | 5.0 mg |
| Sodium starch glycolate | 7.5 mg |
| Magnesium stearate | 1.5 mg |
| Total | 125 mg |

Manufacturing process:

Bicalutamide was mixed with the lactose monohydrate, povidone, and half of the disintegrant (sodium starch glycolate) in an instrumented single-pot granulator (Mi-Mi-Pro, Pro-C-epT). Purified water was added (0.25 ml/g active substance), to obtain a wet granulated mass. The resulting mass was dried using a combination of microwave irradiation (50-200 W), vacuum (below 100 mb) and hot air (40-60° C.).

The dried product was then milled and sieved (through 500 μm mesh) until granules of the required size (below 500 μm) were obtained. The granulate was then mixed with the rest of the disintegrant and lubricant just before compression, that was performed in an eccentric instrumented Korsch EK0 press machine, with round punches of 6 mm diameter, obtaining tablets of 31 N average of resistance to crushing.

Analytical Results:

The dissolution profile of the resulting tablets was tested multiple times by using the paddle method under the following conditions: 900 ml aqueous buffered system (pH 7) with 0.75 (w/v)% SDS using a USP apparatus 2 (paddles) at 50 RPM at 37° C. The results are shown in FIG. 6.

### Example 13

### Form II Capsule composition

The composition of the capsules G is the same that for the tablets and is shown in Table 11

TABLE 11

| Ingredients | Capsules G | |
|---|---|---|
| Bicalutamide | 50.0 | mg |
| Lactose monohydrate | 61.0 | mg |
| Povidone | 5.0 | mg |
| Sodium starch glycollate | 7.5 | mg |
| Magnesium stearate | 1.5 | mg |
| Hard gelatin capsule No. | 4 | |

Same blend used for tablets E was then filled into hard gelatin capsules.

The dissolution profile of the resulting capsules was tested by using the paddle method under the following conditions: 900 ml aqueous buffered system (pH 7) with 0.75 (w/v)% SDS using a USP apparatus 2 (paddles) at 50 RPM at 37° C.

More than 75% of the bicalutamide was dissolved within 30 minutes.

In view of the description of the invention, it will be readily apparent to the worker skilled in the art that the same may be varied in many ways without departing from the spirit of the invention and all such modifications are included within the scope of the present invention as set forth in the following claims.

1. A granulate, comprising at least 50% bicalutamide and at least one pharmaceutically acceptable excipient.

2. The granulate according to claim 4, wherein said granulate comprises 60% to 90% bicalutamide.

3. The granulate according to claim 4, wherein said pharmaceutically acceptable excipient is polyvinylpyrrolidone or a fatty acid ester.

4. The granulate according to claim 4, wherein said granulate further comprises a surfactant.

5. The granulate according to claim 2, wherein said granulate was formed using micronized bicalutamide having an average particle size within the range of 1 to 10 microns and having a specific surface area of at least 3 m²/g.

6. A pharmaceutical composition, comprising the granulate according to claim 5 and optionally an auxiliary excipient.

7. The pharmaceutical composition according to claim 6, wherein said composition is a unit dosage form selected from a capsule and a tablet and at least 40% of said unit dosage form is said bicalutamide.

8. The pharmaceutical composition according to claim 7, wherein said dosage form contains said bicalutamide in an amount from 20 to 200 mg.

9. The pharmaceutical composition according to claim 8, wherein said dosage form exhibits a dissolution profile in vitro such that at 30 minutes at least 75% of the bicalutamide has been released.

10. The pharmaceutical composition according to claim 9, wherein said dosage form exhibits a dissolution profile in vitro such that at 30 minutes at least 90% of the bicalutamide has been released.

11. A solid oral dosage form comprising at least 40% bicalutamide and at least one pharmaceutically acceptable excipient.

12. The solid oral dosage form according to claim 11, wherein said dosage form comprises 50% to 80% of said bicalutamide.

13. The solid oral dosage form according to claim 11, wherein said dosage form exhibits a dissolution profile in vitro such that at 30 minutes at least 75% of the bicalutamide has been released.

14. The solid oral dosage form according to claim 11, wherein said dosage form exhibits a dissolution profile in vitro such that at 30 minutes at least 90% of the bicalutamide has been released.

15. The solid oral dosage form according to claim 11, wherein said dosage form was formed using micronized bicalutamide having an average particle size within the range of 1 to 10 microns and having a specific surface area of at least 3 m²/g.

16. The solid oral dosage form according to claim 15, wherein said dosage form is a tablet that contains at 50 to 150 mg of said bicalutamide.

17. The solid oral dosage form according to claim 15, wherein said dosage form is a capsule that contains at 50 to 150 mg of said bicalutamide.
